(19) United States
(12) Patent Application Publication
(10) Pub. No.: US 2008/0194001 A1
(43) Pub. Date: Aug. 14, 2008

(54) Chimeric Virus for the Treatment of Cancer

(76) Inventor: Edward J. Shillitoe, Fayetteville, NY (US)

Correspondence Address: HOFFMAN WARNICK LLC, 75 STATE STREET, 14TH FLOOR, ALBANY, NY, 12207, US

(21) Appl. No.: 11/817,727

(22) PCT Filed: Mar. 6, 2006
(86) PCT No.: PCT/US06/07908

§ 371 Date: Sep. 4, 2007

Related U.S. Application Data
(60) Provisional application No. 60/660,266, filed on Mar. 10, 2005.
(60) Provisional application No. 60/660,764, filed on Mar. 11, 2005.

(51) Int. Cl. C12N 7/00

(52) U.S. Cl. 435/235.1

(57) ABSTRACT

The invention provides a chimeric virus and a method for its use in the treatment of cancer. In one embodiment, the chimeric virus includes a herpes simplex virus type-1 (HSV-1)/human papillomavirus type-16 (HPV-16) chimera useful in the treatment of an oral or pharyngeal cancer.

### CROSS-REFERENCE TO RELATED APPLICATIONS

This application claims the benefit of co-pending U.S. Provisional Application Nos. 60/660,266, filed 10 Mar. 2005 and 60/660,764, filed 11 Mar. 2005, which are hereby incorporated herein.

### BACKGROUND OF THE INVENTION

1. Technical Field

The invention relates generally to the treatment of cancer, and more particularly, to the treatment of oral and pharyngeal cancers using a chimeric virus.

2. Background Art

Cancers of the oral cavity and pharynx affect approximately 28,000 persons in the United States. Of those affected, approximately 7,000 die each year as a result of their cancer. Currently, the five-year survival rate among those with cancer of the oral cavity or pharynx is 59%, having improved only 5% in the last 25 years.

The use of viruses in the treatment of cancer has been investigated. For example, both adenoviruses and herpes simplex virus type-1 (HSV-1) are known to be toxic, killing their host cells as they replicate. The wild type strains of these viruses, however, are capable of infecting both cancerous and non-cancerous cells. HSV-1, for example, is known to spread to the nervous system, causing encephalitis and, in some cases, death. As a result, strains of such viruses have been developed that replicate only in tumor cells. These are commonly known as “conditionally-replicating viruses” or “oncolytic viruses.”

Oncolytic viruses are generally produced through the deletion of one or more genes associated with viral replication. For example, at least two strains of HSV-1 have been developed. The NV1020 strain, originally developed as a vaccine for HSV-1 contains only one copy of the γ₁ 34.5 neurovirulence gene, has a deletion of the UL56 gene, and a deletion of the promoter region of the UL24 gene. The G207 strain has a deletion of both copies of the γ₁ 34.5 neurovirulence gene and also includes a lacZ gene inserted into the UL39 gene. This insertion interrupts and inactivates the gene, which normally encodes for the large subunit of ribonucleotide reductase, an enzyme required for viral DNA synthesis in nondividing cells.

Both the NV1020 and G207 strains of HSV-1 have been shown to infect human cancer cells xenografted to animal models. In addition, a synergistic effect has been noted when either strain is used in combination with other cancer therapies, such as radiation.

The use of oncolytic viruses in treating cancers is not, however, without defects. As a consequence of their deletions and inactivations, the anti-tumor aggressiveness of oncolytic viruses is typically greatly reduced compared to that of the wild type. It has been observed, for example, that oncolytic viruses are only capable of infecting and destroying tumor cells in the immediate vicinity of the site of administration.

To date, no conditionally-replicating or oncolytic virus has been developed to specifically target oral or pharyngeal cancer cells. To this extent, a need exists for an oncolytic virus that does not suffer from the defects of known strains. More specifically, a need exists for such an oncolytic virus capable of infecting and destroying oral or pharyngeal cancer cells.

### SUMMARY OF THE INVENTION

The invention provides a chimeric virus and a method for its use in the treatment of cancer. In one embodiment, the chimeric virus includes a herpes simplex virus type-1 (HSV-1)/human papillomavirus type-16 (HPV-16) chimera useful in the treatment of an oral or pharyngeal cancer.

A first aspect of the invention provides a method for treating cancer in an individual, the method comprising: administering to the individual an effective amount of a herpes simplex virus type-1 (HSV-1)/human papillomavirus type-16 (HPV-16) chimera.

A second aspect of the invention provides a chimeric virus comprising: deoxyribonucleic acid (DNA) originating from herpes simplex virus type-1 (HSV-1); and DNA originating from human papilloma virus type-16 (HPV-16).

A third aspect of the invention provides a method for the manufacture of a viral chimera, the method comprising: cloning a first portion of a first viral genome under the control of an upstream regulatory region (URR) of a second viral genome; and recombining the first portion of the first viral genome with a second portion of the first viral genome, wherein the second viral genome exhibits a cell specificity different than a cell specificity exhibited by the first viral genome.

The illustrative aspects of the present invention are designed to solve the problems herein described and other problems not discussed, which are discoverable by a skilled artisan.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a partial genomic map of a chimeric virus according to the invention.

FIG. 2 shows a bar graph of the ability of a chimeric virus according to the invention to infect various cell types.

FIG. 3 shows photomicrographs depicting the cytopathic effect of a wild type herpes simplex virus type-1 (HSV-1), a mutant strain of HSV-1, and a chimeric virus according to the invention.

It is noted that the drawings of the invention are not to scale. The drawings are intended to depict only typical aspects of the invention, and therefore should not be considered as limiting the scope of the invention.

### DETAILED DESCRIPTION

As indicated above, the invention provides a chimeric HSV-1/HPV-16 virus (hereinafter “HSPV-1 chimeric virus”) and a method for its use in the treatment of cancer. More particularly, the invention provides an HSPV-1 chimeric virus and a method for its use in the treatment of oral and pharyngeal cancers.

As used herein, an “effective” amount is an amount sufficient to alleviate a symptom associated with a cancer. This may include, for example, a reduction in size of a tumor, an inhibition in the spread of cancerous cells, a reduction in pain or discomfort caused by a tumor or other cancerous growth, prevention of the development of a cancer from a pre-cancerous lesion, and/or reduction in the likelihood of recurrence of a tumor following successful treatment.

As has been already noted, the deletion of one or more genes responsible for viral replication from an oncolytic virus decreases the virus' anti-tumor aggressiveness. Preferably, therefore, an oncolytic virus would include a viral replication gene specific for, or otherwise attenuated to infect, only cancerous cells. In the treatment of oral and/or pharyngeal cancers, it would be advantageous to include a viral replication gene from a virus related to oral or pharyngeal cancer within the genome of a virulent virus, such as HSV-1. One such virus is human papillomavirus type-16 (HPV-16), an oral cancer-related virus. Accordingly, an HSV-1/HPV-16 chimeric virus, such as that of the present invention, would be useful in the treatment of oral and/or pharyngeal cancers. In addition, an HSV-1/HPV-16 chimeric virus according to the invention may be useful in the treatment of any number of cancers within which an HSV-1 virus may grow, including, for example, cervical cancers, skin cancers, breast cancers, and lung cancers.

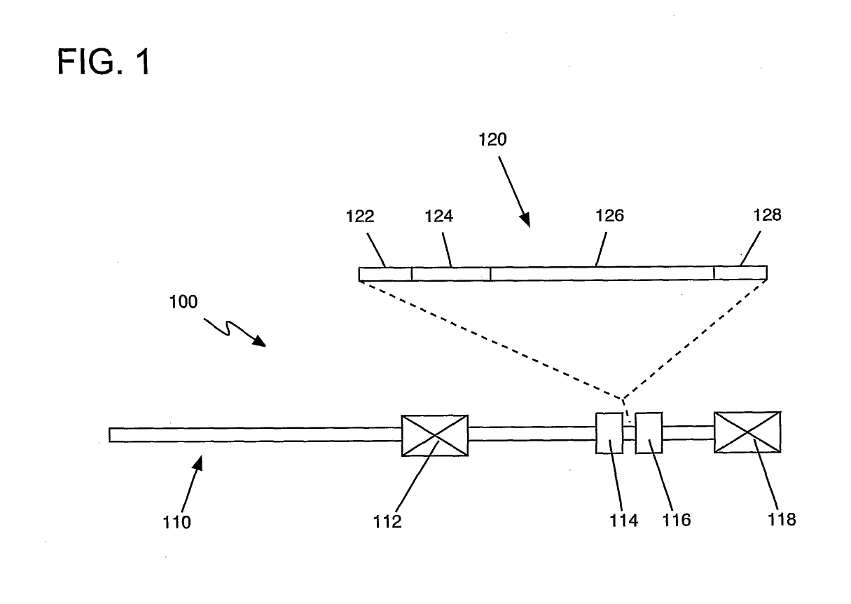

Referring to FIG. 1, a partial map of the genome of the HSPV-1 chimeric virus 100 is shown. Within the HSV-1 DNA 110, both copies of the infected cell polypeptide 4 (ICP4) gene 112, 118 are removed, as in the d120 mutant of HSV-1. Between the Unique Short 9 (US9) gene 114 and the US10 gene 116, a portion of plasmid DNA 120 is inserted via recombination, the plasmid DNA 120 comprising a left intergenic region (LIGR) 122 and a right intergenic region (RIGR) 128 flanking the HPV-16 upstream regulatory region (URR) 124 and a copy of the ICP4 gene 126. That is, the HSPV-1 chimeric virus 100 comprises an ICP4 gene 126 cloned under the control of the HPV-16 URR 124 inserted into mutant HSV-1 DNA lacking both ICP4 genes 112, 118. The cloning and recombination above may be carried out using any known or later-developed method.

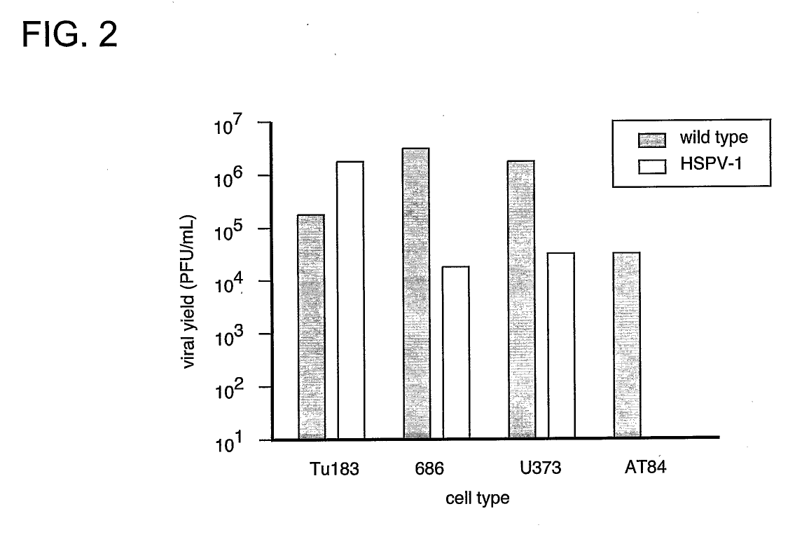

FIG. 2 shows a bar graph of the ability of the wild type HSV-1 and HSPV-1 chimera of the invention to infect various cell types. Cell types included two oral cancer cell lines, TU183 and 686; a neuroblastoma cell line, U373; and a mouse oral cancer cell line, AT84. As can be seen in FIG. 2, the wild type HSV-1 infected all cell types, with the best replication found in the 686 cell line. Wild type HSV-1 also infected the AT84 cell line.

While the replication of the HSPV-1 chimera of the invention was less than that of the wild type HSV-1 in the 686 cell line, it was greater than that of the wild type HSV-1 in the TU183 cell line. More significantly, however, the HSPV-1 chimera of the invention exhibited reduced replication in the U373 neuroblastoma cells and did not infect cells of the AT84 oral cancer cell line at all. These results suggest that the HSPV-1 chimera of the invention may be used to treat cancer in an individual with a reduced risk of infection and destruction of other cells. The fact that the HSPV-1 chimera did not infect cells of the oral cancer cell line AT84 suggest that the host range of the wild type HSV-1 virus, which does infect AT84 cells, has been modified in the HSPV-1 chimera by the HPV-16 promoter.

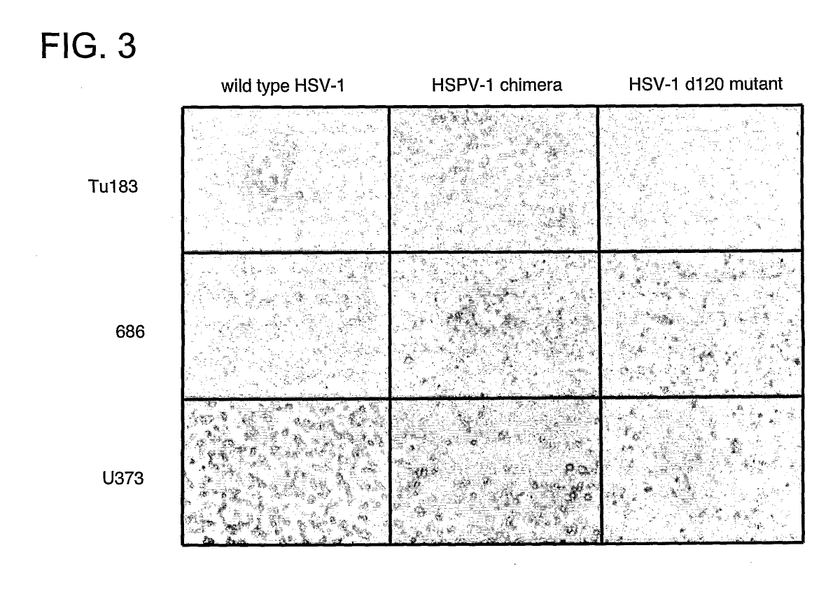

Referring now to FIG. 3, six photomicrographs are shown, representing the cytopathic effect (CPE) of the wild type HSV-1, the HSPV-1 chimera, and the HSV-1 d120 mutant on three cell types, the Tu183 and 686 oral cancer cells and the U373 neuroblastoma cells. Each cell type was infected with each of the three viruses at a concentration of 0.01 PFU/cell and photographed with a 10× objective after 48 hours.

As can be seen in FIG. 3, the HSV-1 d120 mutant, which lacks both copies of the ICP4 gene, exhibited no CPE in any cell type. Both oral cancer cell types, Tu183 and 686, proved susceptible to both the wild type HSV-1 and the HSPV-1 chimera. However, the U373 neuroblastoma cells proved susceptible only to the wild type HSV-1. Significantly, the HSPV-1 chimera exhibited no CPE in the U373 cells. These results also suggest that the HSPV-1 chimera may be used to treat oral cancers without the risk of migration of the virus to cells of the nervous system. Such treatment may include, for example, the injection of an effective amount of the HSPV-1 chimeric virus into known cancerous cells. Other modes of administration include, for example, injection into an artery supplying blood to a tumor and surface applications via lavage, irrigation, or a mouthwash rinse. In addition, such treatment may be combined with other cancer treatments, such as radiotherapy, chemotherapy, and surgical removal of cancerous cells.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A method for treating cancer in an individual, the method comprising:
administering to the individual an effective amount of a herpes simplex virus type-1 (HSV-1)/human papillomavirus type-16 (HPV-16) chimera.

2. The method of claim 1, wherein a portion of the chimera's deoxyribonucleic acid (DNA) originating from HSV-1 lacks at least one gene coding for a viral activator protein.

3. The method of claim 2, wherein the at least one gene codes for infected cell polypeptide 4 (ICP4).

4. The method of claim 3, wherein the chimera lacks both HSV-1 ICP4 genes.

5. The method of claim 2, wherein the chimera includes at least one gene coding for infected cell polypeptide 4 (ICP4) cloned under control of an upstream regulatory region (URR) of the HPV-16 DNA.

6. The method of claim 1, wherein the cancer includes at least one of: a cancer of the oral cavity, a pharyngeal cancer, a cervical cancer, a skin cancer, a breast cancer, and a lung cancer.

7. The method of claim 1, wherein the administering step includes at least one of the following: injection of a tumor, injection into an artery supplying blood to a tumor, lavage, irrigation, and rinsing with a mouthwash.

8. The method of claim 1, further comprising:
administering to the individual at least one of the following: a chemotherapy, a radiation therapy, and surgical removal of cancerous cells.

9. A chimeric virus comprising:
deoxyribonucleic acid (DNA) originating from herpes simplex virus type-1 (HSV-1); and
DNA originating from human papilloma virus type-16 (HPV-16).

10. The chimeric virus of claim 9, wherein the DNA originating from HPV-16 includes at least one regulatory region.

11. The chimeric virus of claim 10, wherein the DNA originating from the HSV-1 includes infected cell polypeptide 4 (ICP4).

12. The chimeric virus of claim 11, wherein the ICP4 gene was cloned under control of an upstream regulatory region (URR) of the HPV-16 DNA.

13. The chimeric virus of claim 11, wherein the DNA originating from HSV-1 does not include an ICP4 gene other than the ICP4 gene cloned under control of the URR of the HPV-16 DNA.

14. A method for the manufacture of a viral chimera, the method comprising:
cloning a first portion of a first viral genome under the control of an upstream regulatory region (URR) of a second viral genome; and
recombining the first portion of the first viral genome with a second portion of the first viral genome,
wherein the second viral genome exhibits a cell specificity different than a cell specificity exhibited by the first viral genome.

15. The method of claim 14, wherein the first viral genome includes the herpes simplex virus type-1 (HSV-1) genome.

16. The method of claim 14, wherein the second viral genome includes the human papillomavirus type-16 (HPV-16) genome.

17. The method of claim 14, further comprising:
inactivating at least one gene coding for a viral activator protein in the first viral genome.

18. The method of claim 17, wherein the first portion of the first viral genome includes the at least one gene coding for a viral activator protein.

19. The method of claim 17, wherein the at least one gene coding for a viral activator protein includes an infected cell polypeptide 4 (ICP4) gene.
